(19) United States
(12) Patent Application Publication
(10) Pub. No.: US 2014/0275505 A1
(43) Pub. Date: Sep. 18, 2014

(54) PROCESS FOR THE PREPARATION OF CERTAIN TRIARYL RHAMNOSE CARBAMATES

(71) Applicant: Dow AgroSciences LLC, Indianapolis, IN (US)

(72) Inventors: James M. RENGA, Indianapolis, IN (US); Anne M. WILSON, Indianapolis, IN (US); Gary D. CROUSE, Noblesville, IN (US)

(73) Assignee: Dow AgroSciences LLC, Indianapolis, IN (US)

(21) Appl. No.: 14/192,425

(22) Filed: Feb. 27, 2014

Related U.S. Application Data
(60) Provisional application No. 61/778,470, filed on Mar. 13, 2013.

(51) Int. Cl. C07H 1/00

(52) U.S. Cl. CPC C07H 1/00
USPC 536/17.4

(57) ABSTRACT

Triaryl rhamnose carbamate insecticides are prepared from triaryl carbamates and the tetrahydropyran-2-ols in good yield without the use of a hydride base.

### CROSS REFERENCE TO RELATED APPLICATIONS

This application claims the benefit of U.S. Provisional Patent Application Ser. No. 61/778,470 filed Mar. 13, 2013, the entire disclosure of which is hereby expressly incorporated by reference.

### BACKGROUND OF THE INVENTION

The present invention concerns an improved process for preparing certain triaryl rhamnose carbamates.

U.S. Pat. No. 8,178,658 describes, inter alia, certain triaryl rhamnose carbamates and their use as insecticides. One of the methods used to prepare such triaryl compounds is by way of a the following 2 step process

[chemical structure]

in which a triaryl amine is reacted with an aryl or substituted aryl chloroformate with a good leaving group to provide a triaryl carbamate followed by reaction with a tetrahydropyran-2-ol to give the triaryl rhamnose carbamate pesticide. However, the reaction of the triaryl carbamate with the tetrahydropyran-2-ol requires the use of a strong hydride base. It would be desirable to have a process in which the triaryl carbamate and the tetrahydropyran-2-ol could be coupled in good yield without the use of a hydride base.

### SUMMARY OF THE INVENTION

The present invention provides such conditions. Thus, the present invention concerns a process for preparing certain triaryl rhamnose carbamates of the Formula (I),

[chemical structure]

wherein

R₁ represents (C₁-C₆) haloalkyl or (C₁-C₆) haloalkoxy, and

R₂ represents (C₁-C₆) alkyl, (C₃-C₆) alkenyl, or (C₃-C₆) alkynyl,

which comprises contacting a triaryl carbamate of Formula (II)

[chemical structure]

wherein

R₁ is as previously defined, and

R₃ represents a phenyl group substituted with one or more substituents selected from the group consisting of F, Cl, Br, I, NO₂ and CN,

with a tetrahydropyran-2-ol of Formula (III)

[chemical structure]

wherein

R₂ is as previously defined,

in an inert organic solvent in the presence of a tertiary amine base and an inorganic base at a temperature from about 20° C. to about 100° C.

### DETAILED DESCRIPTION OF THE INVENTION

Throughout this document, all temperatures are given in degrees Celsius, and all percentages are weight percentages of isolated products unless otherwise stated.

The term “alkyl”, as well as derivative terms such as “haloalkyl” and “haloalkoxy”, as used herein, include within their scope straight chain, branched chain and cyclic moieties. Thus, typical alkyl groups are methyl, ethyl, propyl, butyl, pentyl, hexyl, 1-methylethyl, 1,1-dimethylethyl, 1-methylpropyl, 2-methylpropyl, cyclopropyl, cyclobutyl, cyclopentyl and cyclohexyl. The term “alkenyl”, as used herein, means an acyclic, unsaturated (at least one carbon-carbon double bond), branched or unbranched substituent consisting of carbon and hydrogen, for example, allyl, butenyl, pentenyl or hexenyl. The term “alkynyl”, as used herein, means an acyclic, unsaturated (at least one carbon-carbon triple bond), branched or unbranched substituent consisting of carbon and hydrogen, for example, propargyl, butynyl, pentynyl or hexynyl. The terms “haloalkyl” and “haloalkoxy” includes alkyl or alkoxy groups substituted with from one to the maximum possible number of halogen atoms, all combinations of halogens included. The term “halogen” or “halo” includes fluorine, chlorine, bromine and iodine, with fluorine being preferred.

The present invention concerns a process for preparing certain triaryl rhamnose carbamates of the Formula (I),

[chemical structure]

wherein

R₁ represents (C₁-C₆) haloalkyl or (C₁-C₆) haloalkoxy, and

R₂ represents (C₁-C₆) alkyl, (C₃-C₆) alkenyl or (C₃-C₆) alkynyl,

by reacting a triaryl carbamate of Formula (II)

[chemical structure]

wherein

R₁ is as previously defined, and

R₃ represents a phenyl group substituted with one or more substituents selected from the group consisting of F, Cl, Br, I, NO₂ and CN,

with a tetrahydropyran-2-ol of Formula (III)

[chemical structure]

wherein

R₂ is as previously defined,

in good yield without having to use a hydride base. This is accomplished by performing the reaction in an inert organic solvent in the presence of a tertiary amine base and an inorganic base at a temperature from about 20° C. to about 100° C. The tetrahydropyranols of Formula III consist of approximately a 3:1 mixture of C2 anomers, with the 2R anomer predominating. The initial carbamate product, therefore, consists of a mixture of C2-anomers, formed in the same ratio. Under the conditions described above, this initially formed isomeric mixture undergoes equilibration, leading ultimately to a product that is almost exclusively the (2S) configuration.

R₁ is preferably a (C₁-C₂) fluoroalkoxy group; R₂ is preferably CH₂CH₂CH₃ or CH₂CH═CH₂; and R₃ is preferably para-NO₂ phenyl.

The triaryl carbamates of Formula I are known from U.S. Pat. No. 8,178,658. Approximately a 1:1 molar ratio of the triaryl carbamate and the tetrahydropyran-2-ol may be used, however, molar ratios of about 1.2:1 to about 1:1.2 may also be used. Suitable examples of tetrahydropyran-2-ols include (3R,4R,5S,6S)-3,4,5-trimethoxy-6-methyltetrahydro-2H-pyran-2-ol, (3R,4R,5S,6S)-4-ethoxy-3,5-dimethoxy-6-methyltetrahydro-2H-pyran-2-ol, (3R,4R,5S,6S)-3,5-dimethoxy-6-methyl-4-propoxytetrahydro-2H-pyran-2-ol, and (3R,4R,5S,6S)-4-(allyloxy)-3,5-dimethoxy-6-methyltetrahydro-2H-pyran-2-ol. Currently, it is preferred if (3R,4R,5S,6S)-3,5-dimethoxy-6-methyl-4-propoxytetrahydro-2H-pyran-2-ol or (3R,4R,5S,6S)-4-(allyloxy)-3,5-dimethoxy-6-methyltetrahydro-2H-pyran-2-ol is used.

The reaction is conducted in a wide variety of organic solvents including, for example, halogenated hydrocarbons, e.g. dichloromethane (CH₂Cl₂) and dichloroethane (DCE); aromatic hydrocarbons, e.g., benzene, toluene, or xylenes; polar aprotic solvents, e.g., tetrahydrofuran (THF), acetonitrile (MeCN), methyl tert-butyl ether (MTBE), and mixtures thereof. Currently, it is preferred if MeCN is used.

The reaction requires at least one equivalent of a tertiary amine base, but a 2- to 3-fold excess of tertiary amine base is often recommended. Preferred tertiary amine bases include triethylamine (TEA) and ethyl diisopropylamine (DIPEA).

In addition to the tertiary amine base, the reaction requires the presence of an inorganic base. Typical inorganic bases that can be used include alkali metal and alkaline earth metal carbonates, sulfates and phosphates. Preferred inorganic bases include cesium carbonate (Cs₂CO₃), potassium carbonate (K₂CO₃) and potassium phosphate (K₃PO₄). From 0.1 to 0.5 equivalents of inorganic base is usually sufficient.

The reaction is conducted at a temperature from about 20° C. to about 100° C., with a temperature from about room temperature to about 50° C. being preferred.

In a typical reaction, the triaryl carbamate.hydrochloride (HCl), the tetrahydropyran-2-ol and 0.2 equivalents of inorganic base are mixed in MeCN. About 2 equivalents of tertiary amine base are added and the mixture stirred at ambient temperature until the reaction is completed. The reaction mixture is partitioned between water and a water immiscible solvent such as MTBE. The solvent is evaporated and the isolated product purified by conventional techniques such as flash chromatography.

The following examples are presented to illustrate the invention.

### EXAMPLES

### Example 1

### Preparation of 4-nitrophenyl (4-(1-(4-(perfluoroethoxy)-phenyl)-1H-1,2,4-triazol-3-yl)phenyl)carbamate, HCl

[chemical structure]

To a round bottomed flask equipped with a magnetic stir bar under nitrogen was added 4-nitrophenyl chloroformate (0.544 g, 2.70 mmol) and THF (15 mL) To a separate round bottomed flask under nitrogen was added 4-(1-(4-(perfluoroethoxy)phenyl)-1H-1,2,4-triazol-3-yl)aniline (1 g, 2.70 mmol) and THF (15 mL). The aniline solution was added via syringe to the chloroformate solution, and after a small amount of bubbling (no heat generation) a thick white precipitate began to form within 5 minutes (min). After stirring the reaction mixture for 1 hour (h) at room temperature, the off-white solid was filtered, washed with hexanes and air-dried to give the title compound (1.48 g, 96%): mp 170-174° C.; ¹H NMR (400 MHz, DMSO-d₆) δ 10.71 (s, 1H), 9.39 (s, 1H), 8.39-8.25 (m, 2H), 8.16-8.00 (m, 4H), 7.67 (d, J=8.6 Hz, 2H), 7.64-7.51 (m, 4H); ¹³C NMR (101 MHz, DMSO-d₆) δ 161.77, 155.41, 150.49, 146.06, 144.62, 143.71, 139.50, 135.82, 126.92, 125.23, 125.13, 123.13, 122.93, 121.07, 118.69, 118.66, 99.48; ESIMS m/z 534 ([M+]).

### Example 2

### Preparation of (2S,3R,4R,5S,6S)-3,5-dimethoxy-6-methyl-4-propoxytetrahydro-2H-pyran-2-yl (4-(1-(4-(perfluoroethoxy)phenyl)-1H-1,2,4-triazol-3-yl)phenyl)carbamate

[chemical structure]

To a magnetically stirred solution of (3R,4R,5S,6S)-3,5-dimethoxy-6-methyl-4-propoxytetrahydro-2H-pyran-2-ol (4.12 g, 17.6 mmol) and 4-nitro-phenyl (4-(1-(4-(perfluoroethoxy)phenyl)-1H-1,2,4-triazol-3-yl)phenyl)carbamate, HCl (10.05 g, 17.57 mmol) in MeCN (100 mL) was added freshly ground potassium phosphate, tribasic (K₃PO₄; 0.746 g, 3.51 mmol). To the resulting off-white mixture was added DIPEA (6.12 mL, 35.1 mmol) and the resulting bright yellow mixture was stirred at room temperature for 6 h. The mixture was partitioned between MTBE (400 mL) and water (100 mL), and the phases were separated. The organic phase was successively washed with water (3×100 mL), aqueous sodium hydroxide (NaOH, 1 N, 100 mL), and water (100 mL). The organic phase was dried over magnesium sulfate (MgSO₄), filtered, and concentrated to give an orange foam. The foam was purified by flash column chromatography (3:3:3:1 hexane:ethyl acetate (EtOAc):CH₂Cl₂:acetone) to give product fractions that were orange in color. The combined fractions were treated with decolorizing charcoal, filtered, and concentrated to give the title compound as a yellow solid (7.01 g, 63.3%): mp 152-154° C.; ¹H NMR (400 MHz, CDCl₃) δ 8.57 (s, 1H), 8.23-8.11 (m, 2H), 7.86-7.76 (m, 2H), 7.54 (d, J=8.3 Hz, 2H), 7.43-7.36 (m, 2H), 6.85 (s, 1H), 6.20 (d, J=2.0 Hz, 1H), 3.74-3.48 (m, 13H), 3.21 (t, J=9.4 Hz, 1H), 1.75-1.64 (m, 3H), 1.37-1.30 (m, 3H), 1.30-1.22 (m, 2H), 0.99 (t, J=7.4 Hz, 3H); ¹⁹F NMR (376 MHz, CDCl₃) δ −85.90, −87.86; ESIMS m/z 631 ([M+]).

### Example 3

### Preparation of (2S,3R,4R,5S,6S)-3,5-dimethoxy-6-methyl-4-propoxytetrahydro-2H-pyran-2-yl (4-(1-(4-(perfluoroethoxy)phenyl)-1H-1,2,4-triazol-3-yl)phenyl)carbamate

[chemical structure]

To a magnetically stirred solution of (3R,4R,5S,6S)-3,5-dimethoxy-6-methyl-4-propoxytetrahydro-2H-pyran-2-ol (0.434 g, 1.85 mmol) and 4-nitro-phenyl (4-(1-(4-(perfluoroethoxy)phenyl)-1H-1,2,4-triazol-3-yl)phenyl)carbamate, HCl (0.992 g, 1.85 mmol) in MeCN (8.5 mL) was added Cs₂CO₃, (0.103 g, 0.315 mmol). To the resulting mixture was added DIPEA (0.645 mL, 3.70 mmol) and the resulting yellow mixture was stirred at room temperature for 1.5 h. The mixture was partitioned between CH₂Cl₂ and water, and the phases were separated. The organic phase was washed with saturated aqueous sodium bicarbonate (NaHCO₃, 3×30 mL), dried over MgSO₄, filtered, and concentrated to give a yellow foam. The foam was purified by flash column chromatography (3:3:3:1 hexane:EtOAc:CH₂Cl₂:acetone) to give the title compound as a yellow solid (0.843 g, 72%):

¹H NMR (400 MHz, CDCl₃) δ 8.57 (s, 1H), 8.23-8.11 (m, 2H), 7.86-7.76 (m, 2H), 7.54 (d, J=8.3 Hz, 2H), 7.43-7.36 (m, 2H), 6.85 (s, 1H), 6.20 (d, J=2.0 Hz, 1H), 3.74-3.48 (m, 13H), 3.21 (t, J=9.4 Hz, 1H), 1.75-1.64 (m, 3H), 1.37-1.30 (m, 3H), 1.30-1.22 (m, 2H), 0.99 (t, J=7.4 Hz, 3H); ESIMS m/z 631 ([M+]).

### Example 4

### Preparation of (2S,3R,4R,5S,6S)-3,5-dimethoxy-6-methyl-4-propoxytetrahydro-2H-pyran-2-yl (4-(1-(4-(trifluromethyl)phenyl)-1H-triazol-3-yl)phenyl)carbamate

[chemical structure]

Step 1. 4-[1-(4-Trifluoromethylphenyl)-1H-[1,2,4]triazol-3-yl]aniline was prepared by coupling 1-(4-nitrophenyl) 1,2,4-triazole with 4-iodobenzotrifluoride according to conditions described in Crouse, et. al., U.S. Pat. No. 8,178,658, to generate 3-(4-nitrophenyl)-1-(4-(trifluoromethyl)phenyl)-1H-1,2,4-triazole as a tan solid: mp 161-162° C.; ¹H NMR (300 MHz, CDCl₃) δ 8.60 (s, 1H), 8.40-8.35 (m, 4H), 7.91 (d, J=7.8 Hz, 2H), 7.83 (d, J=8.6 Hz, 2H); EIMS m/z 335 ([M+1]±). Catalytic hydrogenation of the nitro group using palladium on carbon (Pd/C, 5%) in methanol (MeOH) provided the corresponding aniline as a light tan solid: mp 178-180° C.; ¹H NMR (400 MHz, CDCl₃) δ 8.60 (s, 1H), 8.00 (d, J=8.6 Hz, 1H), 7.87 (d, J=7.8 Hz, 2H), 7.80-7.73 (m, 2H), 6.76 (d, J=8.6 Hz, 1H), 3.88 (s, 2H); EIMS m/z 304.3 ([M+]).

### Step 2. (2S,3R,4R,5S,6S)-3,5-dimethoxy-6-methyl-4-propoxytetrahydro-2H-pyran-2-yl (4-(1-(4-(trifluoromethyl)phenyl)-1H-1,2,4-triazol-3-yl)phenyl)-carbamate

To a magnetically stirred solution of 4-(1-(4-(trifluoromethyl)phenyl)-1H-1,2,4-triazol-3-yl)aniline (5.0 g, 16.4 mmol) in THF (20 mL) was added 4-nitrophenyl carbonochloridate (3.31 g, 16.4 mmol). A solid formed rapidly, and this was filtered and air-dried to give 4-nitrophenyl (4-(1-(4-(trifluoromethyl)-phenyl)-1H-1,2,4-triazol-3-yl)phenyl)carbamate.HCl (6.76 g, 81%). A portion of this salt (2.10 g, 4.16 mmol) was slurried in dry MeCN (20 mL) and stirred magnetically while (3R,4R,5S,6S)-3,5-dimethoxy-6-methyl-4-propoxytetrahydro-2H-pyran-2-ol (1.05 g, 4.47 mmol) was added, followed by Cs₂CO₃ (0.25 g, 0.77 mmol). To the resulting mixture was added DIPEA (1.20 g, 9.28 mmol) and the solution was allowed to stir at ambient temperature for 4 h. The mixture was partitioned between diethyl ether (50 mL) and a saturated aqueous NaHCO₃ solution (50 mL). The organic layer was dried and concentrated to a yellow gum, which was crystallized by stirring in ether-hexanes (1:3) to furnish the title compound (2.18 g, 82%): mp 188-191° C.; ¹H NMR (400 MHz, CDCl₃) δ 8.65 (s, 1H), 8.17 (d, J=8.7 Hz, 2H), 7.95-7.85 (m, 2H), 7.80 (d, J=8.5 Hz, 2H), 7.61-7.47 (m, 2H), 6.85 (s, 1H), 6.20 (d, J=2.0 Hz, 1H), 3.76-3.44 (m, 11H), 3.22 (t, J=9.4 Hz, 1H), 1.75-1.60 (m, 2H), 1.33 (d, J=6.2 Hz, 3H), 0.98 (t, J=7.4 Hz, 3H); ESIMS m/z 565 ([M+]).

What is claimed is:

1. A process for preparing triaryl rhamnose carbamates of the Formula (I), [chemical structure]
wherein
R₁ represents (C₁-C₆) haloalkyl or (C₁-C₆) haloalkoxy, and
R₂ represents (C₁-C₆) alkyl, (C₃-C₆) alkenyl or (C₃-C₆) alkynyl,
which comprises contacting a triaryl carbamate of Formula (II) [chemical structure]
wherein
R₁ is as previously defined, and
R₃ represents a phenyl group substituted with one or more substituents selected from the group consisting of F, Cl, Br, I, NO₂ or CN,
with a tetrahydropyran-2-ol of Formula (III) [chemical structure]
wherein
R₂ is as previously defined,
in an inert organic solvent in the presence of a tertiary amine base and an inorganic base at a temperature from about 20° C. to about 100° C.

2. The process of claim 1 in which R₁ is a (C₁-C₂) fluoroalkoxy group.

3. The process of claim 1 in which R₂ is CH₂CH₂CH₃ or CH₂CH═CH₂.

4. The process of claim 1 in which R₃ is para-NO₂ phenyl.

5. The process of claim 1 in which the tertiary amine base ethyl diisopropylamine.

6. The process of claim 1 in which the inorganic base is cesium carbonate.

7. The process of claim 1 in which the inorganic base is potassium phosphate (tribasic).

8. The process of claim 1 in which the inert organic solvent is acetonitrile.
